(19) United States
(12) Patent Application Publication
(10) Pub. No.: US 2026/0199118 A1
(43) Pub. Date: Jul. 16, 2026

(54) 3D PRINTED SPLINT OR CAST FOR AN ARTICULATING JOINT

(71) Applicant: Dimension Ortho Inc., Philadelphia, PA (US)

(72) Inventors: Michael RIVLIN, Philadelphia, PA (US); Pedro K. BEREDJIKLIAN, Philadelphia, PA (US); Ashkan SEDIGH, Philadelphia, PA (US); Alexander R. VACCARO, Philadelphia, PA (US); Michael J. SILESKI, Philadelphia, PA (US)

(21) Appl. No.: 19/135,893

(22) PCT Filed: Dec. 15, 2023
(86) PCT No.: PCT/US2023/084299

§ 371 Date: Jun. 5, 2025

Related U.S. Application Data
(60) Provisional application No. 63/432,765, filed on Dec. 15, 2022.

(51) Int. Cl. A61F 5/02; A61F 5/01; G16H 40/63

(52) U.S. Cl. CPC A61F 5/022; A61F 5/0123; G16H 40/63; A61F 2005/0155; A61F 2005/0167

(57) ABSTRACT

A splint, brace, or any other orthosis for supporting a patient's limb having an articulating joint includes first and second portions configured to connect to the patient's limb, a latching mechanism constructed of a strap, elastic material or fabric to connect the first and second portions and a lattice shape or holes placed automatically or manually by design software on first and second portions to provide air circulation. The first portion being configured for mounting to the patient's limb at a first side of the articulating joint and the second portion configured for mounting to the patient's limb at a second side of the articulating joint.

### CROSS-REFERENCE TO RELATED APPLICATION

The present application claims the benefit of U.S. Provisional Ser. No. 63/432,765 , filed on Dec. 15, 2022 and titled, “3D Printed Splint/Cast and related Method,” the entire contents of which is incorporated herein by reference in its entirety.

### BACKGROUND OF THE INVENTION

Splinting is a short-term method to immobilize joints to treat musculoskeletal abnormalities or injuries. Splinting has various applications, but is commonly used to stabilize a fracture, sprain or strain before or after an operation. Splinting is used to immobilize an acute or occult fracture. Moreover, splinting may be used to protect against severe soft tissue injuries or to facilitate partial immobilization for minor soft tissue injuries. Splinting can also be utilized in immobilizing and treatment of joint instability and peripheral neuropathy, as well as for therapy purposes to maintain orientation and integrity of a patient's joint.

The conventional method of splinting is to utilize and form thermoplastic sheets around the injured area or to utilize conventional sized splints for particular joints. However, splinting can also be performed with other materials, such as fiberglass or plaster. In the thermoplastic formation method, an outline of the injured limb is prepared and after heating the sheet, the pliable sheet can be molded around the target area. This method is not only time-consuming but is also not applicable where urgent service is required. On the other hand, choosing the appropriate type and size of splint or brace, collectively known as orthoses, is paramount for the proper treatment of many conditions. Sizing for a prefabricated wrist splint would require an in-person office visit or a trial-and-error approach for ordering a splint from an online or even a physical vendor. The preferred invention addresses the shortcomings of known splints and splinting methods.

### BRIEF SUMMARY OF THE INVENTION

A splint, brace, cast or other orthosis includes a latching mechanism constructed with a strap. The strap is constructed with an elastic material, a fabric, such as neoprene, Velcro or hook and loop material, a spring mechanism, a pin, or similar mechanical mechanisms, that is configured to interlock multiple surfaces or sections of the splint, brace, cast or other orthosis. The splint, brace, cast or other orthosis also includes a lattice shape or holes placed automatically or manually by design software on a surface of the splint, brace, cast or other orthosis. The lattice shape or holes are configured to provide adequate air circulation to the patient's skin while the splint, brace, cast or other orthosis is mounted to the patient's body.

In another aspect, a method of construction of a splint, brace, cast or other orthosis includes capturing a three-dimensional (“3D”) geometry of a patient's limb in a clinical position with a 3D scanner system. The 3D scanner system performing the scan of the patient's limb in a relatively short scanning time. The 3D scanner includes a Lidar sensor, a red/blue/green wavelength (“RGB”) camera and/or laser technology to capture the 3D geometry of the patient's limb in high resolution. A 3D software is configured to design the splint, brace, cast or other orthosis based on the 3D scanned limb. The software is automated or manual with features, such as a customized latch or locking mechanism, lattice design, name, art engravement, limb position, offsetting to compromise swelling, and mesh deformations to adjust the joint's angle (flexion, extension, etc.).

Moreover, the splint software may enable the user to optimize the design and features (acceleration, elevation, velocity, force sensor, gyroscope, pressure sensor, displacement sensor, etc.) implementation based on a machine learning algorithm and required boundary conditions as biomechanical forces.

The latching mechanism of the splint of the preferred embodiments may include neoprene, Velcro, hook and loop material, a spring mechanism, a pin or similar mechanical mechanism. The preferred latching mechanism may be comprised of elastic mesh straps that can be fitted under the splint, wherein the elastic mesh straps include holes and biocompatible material to avoid skin reactions. The latching mechanism may be configured for customization to include the patient's name, company logo, color, sizing, detachable logo, modular indicators at any spot on the splint or other custom marks or attributes.

In another preferred aspect, the splint is constructed based on a model developed using a 3D scanner system to capture a 3D geometry of the limb in the clinical position and in a short scanning time. The 3D scanner may include a Lidar sensor, a red-blue-green (“RGB”) camera and laser technology to capture the patient's limb in high resolution. The images collected from the 3D scanner are preferably transmitted to 3D software that may be configured to design the splint on the 3D model of the scanned limb. The 3D software is configured for automated or manual operation with features such as a customized latching mechanism, lattice design, name, art engravement, limb position, offsetting to compromise swelling, and mesh deformations to adjust the articulating joint's angle (flexion, extension, etc.). A 3D manufacturing system that machines or prints the designed splint with 3D printing or subtractive technology, computer aided design/computer aided manufacturing (“CAD/CAM”), or hybrid systems may be utilized to construct the final splint.

In an additional aspect, the splint may be comprised of modular parts that are smoothly detachable for applications such as electrode implementation, writing, sport, and clinical evaluation. The modular parts may be detached smoothly in the upper extremity as a modular thumb on hand splint. The modular parts may be interchangeable and configured to be temporarily or permanently removed or attached from each other.

The splint may include breakaway mechanical joints, hinges, and latches to provide a range of motion for the joint during an immobilization period and the breakaway mechanical joints may be constructed of any center of rotation and multi-range of movement mechanism.

In a further aspect, the customized splint may include a hinge or adjustable mechanical range-limiting device for active sports players, cyclists, or other applications to immobilize and protect the joint from further injuries, such as with applications in the lower extremity. The customized splint may lock or block the range of the motion of a particular join by a specific period which is configured for control by remote technology, such as a mobile device application or internet of things (“IoT”) platform, screen on the device or physical controlling options. The customized splint may include hinges, locks, and/or latching mechanisms that are controlled automatically or manually by the design software or central processor. The customized splint may constrain a specific degree of motion, dynamic movement, or direction in sport, medical, space, military or aerospace applications for the joint.

In another aspect, the preferred splint may increase or decrease pressure on the immobilized area with an adjustable fluid/air pressure bladder in the lining bladder of the splint, such as within first and second shells or portions of the splint. The splint may be configured to apply pressure on the immobilizing area hydraulically or with fluidic control to adjust joint angle in medical applications such as diabetic, weak or paralyzed patients or military, space and aerospace applications. The splint may be configured to control a temperature on the immobilizing area with cooling or heating pads manually or in an automated way or may be adapted to include a flow-through heating/cooling system through an embedded channel system in the shells. The splint may include a hydraulic or other machine assist for articulating joints with a pre-set arc of motion that can be adjusted manually or automatically or through a remote system, such as the central processor. The system may also include a pulley and therapeutic bands/springs that are utilized to support the joint and/or to facilitate therapy and strengthening or improving flexibility of the joint. A mechanical or electrical actuator may be utilized for modular active and passive motion in a programmed range of motion for the splint, wherein the programmed range of the motion can be programmed remotely with an application, IoT platform, on-screen control or via the central processor.

In an additional aspect, the utilized sleeve/liner of the splint is automatically or manually customized or mass-customized based on the scanned limb or a general mass-customization guide. The software utilized to generate the 3D model of the patient's limb may also generate a finite-element (“FE”) based design to optimize the material and develop required mechanical strength on the joint for various applications such as sports, aerospace, military, etc. The splint may be mass customized into multiple sizes based on demographic and geometrical limb data selected automatically through artificial intelligence (“AI”).

In a further aspect, the scanner system of the preferred embodiment is configured to utilize pattern recognition and machine learning algorithms to adjust cutout/lattice structure in the splint based on wounds, scars, incisions and other anatomical obstructions. The scanner system may include a hinged apparatus configured for accounting for an axis of rotation, wherein the scanner system automatically places the hinged apparatus in a center of rotation of the joint based on the 3D scanned file with machine vision and machine learning algorithms. The hinged apparatus may be configured for operation with a patient's joint such as an elbow, wrist, finger, hip, knee or ankle.

In another aspect, a platform designs and implements the mechanical or electrical actuator that may be applied to the splint and a customized or mass-customized parts and holders, mechanical shafts, or related modules in a corrected anatomical angle and alignment. The scanner system may be configured to perform a correction manually or with a machine-learning algorithm for decision-making.

In a further aspect, the mechanical or electrical actuator of the preferred splint may include related modules, including shafts, a mechanical hinge, limb holders, straps, and locks, wherein the shafts, mechanical hinge, limb holders, straps and locks are customized or predicted by a mass-customized algorithm based on the input 3D scan. A customized or preferred size from mass customization is chosen or may specifically be manufactured. The splint may be comprised of a spine brace having a goniometer, accelerometer, gyroscope, pressure sensor, displacement sensor, piezo sensor or a wearable force sensor for fitting and tracking.

In an additional aspect, the 3D software of the preferred system may include an automated sizing of a temporary splint from an array of available pre-manufactured sizes. The customized splint may be comprised of a neck/torso/back brace, wherein the neck/torso/back brace includes a cable system that runs parallel to a spine of the patient or in an off-axis direction. The cable system may be configured to allow a predetermined angle of bending in the modular design to facilitate limited movement of the patient to promote healing. The splint may be fitted with sensors, including a goniometer, an accelerometer, a gyroscope, a pressure sensor, a displacement sensor, microfluidic devices, and other electro-mechanical or biological/chemical sensors, including the wearable sensors, for data collection. The sensors may be manually or automatically placed in an optimal position of the splint for optimal collection of data and the collected data is preferably transmitted to the central processor.

In another aspect, the splint includes a joint motion in an articulating portion of the splint that generates feedback through sensors potentially including a goniometer, an accelerometer, a gyroscope, a pressure sensor, a displacement sensor, microfluidic devices, and other electro-mechanical or biological/chemical sensors, including wearable sensors to collect joint motion data and record joint position, angular and linear velocities/acceleration, torque and other parameters. The data from these sensors is collected and preferably transmitted to the central processor. A biomechanical feedback may be analyzed based on the collected data, including impact, torsion, flexion, compression and related parameters to a target spot and the analyzed biomechanical feedback may be transmitted to a physician, therapist, or healthcare system. The biomechanical feedback may be analyzed including impact, torsion, flexion, compression and related parameters to a target spot and sent to a physician, therapist, or healthcare system. The force sensors may manually or automatically placed with a pre-trained system onto the splint for collecting force data while the patient is in therapy or while they generally go about their daily lives.

A hydraulic or other machine assist may be utilized with the splint for articulating joints with preset arc of motion that can be adjusted. A hinged apparatus may also be employed on the splint (i.e. elbow or leg) accounting for axis of rotation that the AI/Machine learning automatically places in the right center of rotation based on the 3D scan and the data transmitted to the central processor. The splint may also employ pulleys, bands and springs for additional therapeutic purposes. An actuator/motor addition may be utilized with the splint for modular assist/active assist and passive range of motor functionalities (AI/ML) that places these modules and locks in the predicted anatomical angle and alignment related to the patient's limb and, specifically, the joint. The preferred splint may also include a pressure sensor that is utilized to monitor snugness and fit of the splint relative to the patient's limb, such as for a spine splint.

In an additional aspect, the central processor may provide feedback from the data collected from the sensors related to joint motion parameters including acceleration, elevation, velocity, force, orientation, pressure, displacement and related parameters. The data may be utilized by the central processor to assist and adjust the mechanical shaft pressure, flow rate, and range of motion or provide a new splint or orthotic design to meet requirements based on a decision-making algorithm and adjust the required control parameters.

In another aspect, the scanner system may generate a design based on the feedback from the data collected (acceleration, elevation, velocity, force, orientation, pressure, displacement, and related parameters) to update a finite-element analysis parameters including boundary conditions, tensor fibers, and related parameters and optimize a material, shape or lattice of the splint.

In a further aspect, the preferred system may be directed to an automatic scan with pattern recognition and adjustment of cutout/lattice structure based on wounds, scars, incisions and other anatomical obstructions that may be detected by the central processor based on collected images of the patient's limb.

The latching mechanism may be comprised of a neoprene, Velcro or hook and loop material, a spring mechanism, a pin, or similar mechanical mechanisms that may be incorporated in the splint, brace or any other orthosis. The latching mechanism may be comprised of elastic mesh straps that can be fitted under the splint, brace, or any other orthosis, wherein the elastic mesh straps include holes and biocompatible material to avoid any skin reaction of the patient's skin under and proximate to where the splint, brace or any other orthosis is mounted. The latching mechanism may be configured to be customized to the patient's name, company logo, color, sizing, detachable, or modular at any spot on the splint, brace, or any other orthotic splint.

The splint, brace or other orthosis is preferably constructed by a 3D scanner system to capture a 3D geometry of the limb in the clinical position and in a short scanning time. The 3D scanner includes a Lidar sensor, RGB camera, and laser technology to capture the patient's limb in high resolution. A 3D software is configured to design the splint, brace, or any other orthosis based on the 3D scanned limb. The 3D software is configured for automated or manual operation with features such as a customized latching mechanism, lattice design, name, art engravement, limb position, offsetting to compromise swelling, and mesh deformations to adjust the articulating joint's angle (flexion, extension, etc.). A 3D manufacturing system of the preferred invention machines or prints the designed splint, brace, or any other orthosis with 3D printing or subtractive technology, CAD/CAM, or hybrid systems. The preferred system may include an automated sizing module for a temporary splint that is selected from an array of available pre-manufactured sizes.

The splint, brace, or any other orthosis may comprise modular parts to be smoothly detachable for applications such as electrode implementation, writing, sport, and clinical evaluation. The splint, brace, or other orthosis preferably comprises modular parts to be detached smoothly in the upper extremity as a modular thumb on hand splint, brace, or orthosis. The splint, brace, or any other orthosis may include breakaway mechanical joints, hinges, and latches to provide the required range of motion during the immobilization period, wherein the breakaway mechanical joints are constructed of any center of rotation and multi-range of movement mechanism. The customized splint, brace, or any other orthosis with applications in the lower extremity may include a hinge for active sports players, cyclists, or other applications to immobilize and protect the joint from any further injuries while allowing articulation at the joint so that the patient maintains the ability to move the joint. The customized splint, brace, or any other orthosis may lock the range of motion of the joint by a specific period to further protect the joint from further injury, such as overextension, flexion or articulation. The hinges, locks, and latching mechanism of the customized splint, brace, or any other orthosis are preferably implemented automatically or manually by the design software during manufacture. The customized splint, brace, or any other orthosis may constrain a specific degree of motion, dynamic movement, or direction in sport, medical or space applications to further support or limit further damage to the joint.

The splint, brace, or any orthosis may be configured to increase or decrease pressure on the immobilizing area wherein the splint, brace or other orthosis is mounted to the patient with adjustable fluid/air pressure in a lining bladder of the device. The splint, brace, or any other orthosis may be configured to apply pressure on the immobilizing area proximate the joint, hydraulically or with fluidic control in medical applications such as diabetic patients or space costumes. The splint, brace, or any other orthosis may be configured to control a temperature on the immobilizing area proximate the joint with cooling or heating pads manually or battery-powered. The utilized sleeve/liner of the device may be customized or mass-customized based on the scanned limb or general mass-customization guide of the preferred invention. A preferred software generates a finite element (“FE”) based design to optimize the material and enhance required mechanical strength on the joint for various applications such as sports, space, military, etc. The splint, brace, or orthosis is preferably mass customized into multiple sizes based on demographic and geometrical limb data stored in a central server.

The preferred system may include an automatic scan with pattern recognition and adjustment of cutout/lattice structure based on wounds, scars, incisions and other anatomical obstructions that are encountered on the patient's appendage. The preferred system may also incorporate a hydraulic or other machine assist for articulating joints utilizing the first and second mounted to the patient's appendage with preset arc of motion that can be adjusted by the clinician or patient.

The preferred system may include a hinged apparatus on the splint that spans the joint (i.e. elbow or leg) accounting for an axis of rotation that the AI/Machine learning automatically places in the right center of rotation based on the 3D scan. The splint, brace or any other orthosis may also include a pulley addition for therapeutic bands/springs. The preferred system may include an actuator/motor addition for modular assist/active assist and passive range of motor functionalities (AI/ML) that places these modules and locks in the predicted anatomical angle and alignment relative to the joint when mounted to the patient. The splint or brace may be adapted or configured as a spine splint that includes a pressure sensor for proper fit/snugness when mounted to the patient.

The preferred method may include automated sizing of a temporary splint or brace or modular orthosis from an array of available pre-manufactured sizes. The preferred splint or brace may be configured as a neck/torso/back splint, where a cable system that runs parallel to the spine or in an off axis direction allows a predetermined angle of bending in the modular design.

Briefly stated, the preferred invention may be directed to a spine splint for supporting a patient's spine and facilitating limited movement of the patient's spine. The spine splint includes a series of interconnected ring portions including a first ring portion and a second ring portion. The first ring portion is configured to articulate relative to the second ring portion. The first ring portion is movably engaged to the second ring portion by a pin slidably positioned within a sliding track thereby mimicking a natural articulation of the patient's spine.

### BRIEF DESCRIPTION OF THE SEVERAL VIEWS OF THE DRAWINGS

The foregoing summary, as well as the following detailed description of preferred embodiments of the 3D printed splint or cast, 3D scanner, related instruments and implants and related methods of the present application, will be better understood when read in conjunction with the appended drawings. For the purposes of illustrating the 3D printed cast, splint, brace or other orthosis, there are shown in the drawings preferred embodiments. It should be understood, however, that the application is not limited to the precise arrangements and instrumentalities shown. In the drawings:

FIG. 1A illustrates a bottom plan view of a splint or brace constructed by a 3D printer and preferred method in accordance with a first preferred embodiment of the present invention;

FIG. 1A illustrates a side perspective view of a finger splint with mechanical joints to implement sensors and specify a range of the motion for a joint in accordance with a first preferred embodiment of the present invention;

FIG. 1B illustrates a rear perspective view of the finger splint of FIG. 1A;

FIG. 1C illustrates a side elevational view of the finger splint of FIG. 1A connected to an additional splint portion or shell with two mechanical joints to implement sensors and interlock a range of motion;

FIG. 1D illustrates a side elevational view of the splint of FIG. 1C with a component removed for clarity;

FIG. 2A illustrates a front perspective view of a motorized splint with one joint and sensors (force, gyroscope, accelerometer, speed) to provide passive or assistive motion in accordance with a second preferred embodiment of the present invention;

FIG. 2B illustrates a side elevational view of the splint of FIG. 1A;

FIG. 2C illustrates a rear perspective view of the splint of FIG. 1A;

FIG. 3 illustrates a side elevational view of a splint or brace with a hydraulic assist, sensors and a processing unit in accordance with a third preferred embodiment of the present invention;

FIG. 4 illustrates a front elevational view of a spine splint or brace for a patient's spine with sensors in accordance with a fourth preferred embodiment of the present invention; and FIG. 5 illustrates a front elevational view of a fourth ring portion of the spine splint of FIG. 4.

### DETAILED DESCRIPTION OF THE INVENTION

Certain terminology is used in the following description for convenience only and is not limiting. Unless specifically set forth herein, the terms “a,” “an” and “the” are not limited to one element but instead should be read as meaning “at least one.” The words “right,” “left,” “lower” and “upper” designate directions in the drawings to which reference is made. The words “inwardly” or “distally” and “outwardly” or “proximally” refer to directions toward and away from, respectively, the patient's body, or the geometric center of the preferred splint or cast and related parts thereof. The words, “anterior,” “posterior,” “superior,” “inferior,” “lateral” and related words and/or phrases designate preferred positions, directions and/or orientations in the human body to which reference is made and are not meant to be limiting. The word “splint” and related words and/or phrases refer generally to a splint, cast, shell, orthosis or other device designed to support a patient's limb, particularly an articulating joint, to encourage proper alignment of the limb, to limit the range of motion of a joint, to assist stability of the joint or to otherwise protect and promote healing of the limb. The terminology includes the above-listed words, derivatives thereof and words of similar import.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

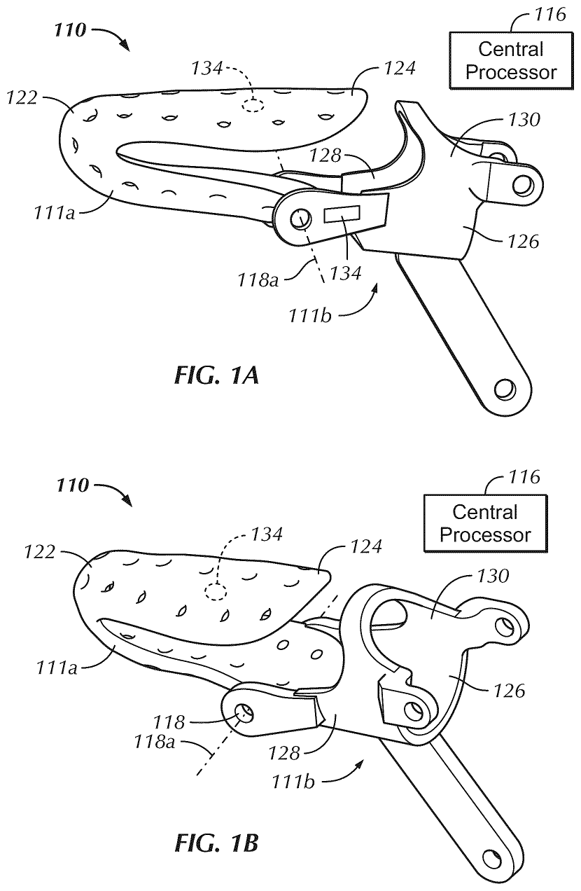

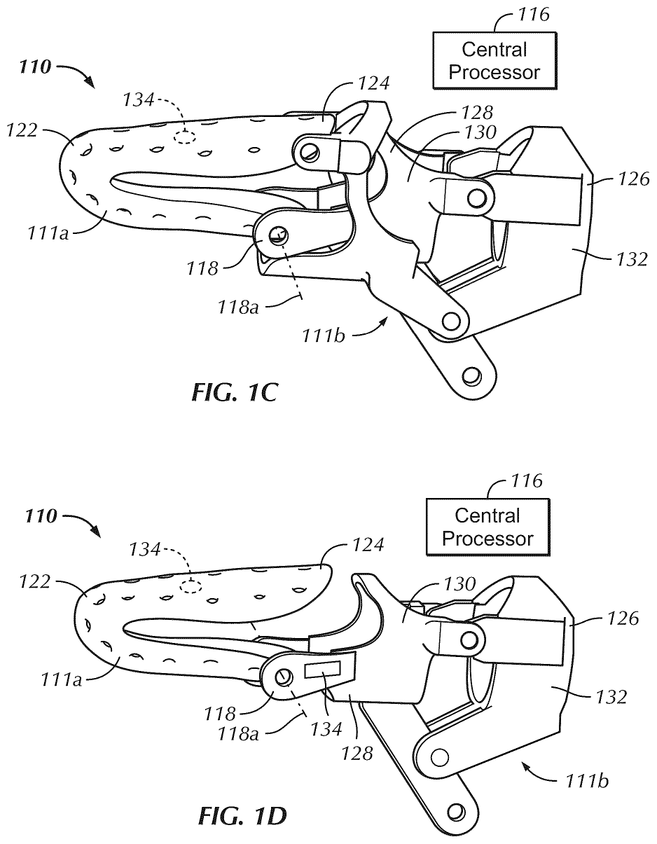

Referring to FIGS. 1A-3, first, second and third preferred embodiments of a splint, brace, or any other orthosis, generally designated 110, 210, 310, for supporting a patient's limb having a first bone, a second bone and an articulating joint between the first and second bones, is described herein. The articulating joint may be a knee, finger, wrist, neck, elbow, shoulder, ankle, toe, back or other articulating joint of the patient. The preferred splint 110, 210, 310 includes first and second portions or shells 111a, 111b, 211a, 211b, 311a, 311b configured to connect to the patient's limb with the first shell 111a, 211a, 311a at a first side of the joint and the second shell 111b, 211b, 311b at a second side of the joint. The preferred splints 110, 210, 310 also include a latching mechanism constructed of first and second bladders 320a, 320b, a strap, elastic material or fabric to connect the first and second shells 111a, 111b, 211a, 211b, 311a, 311b, having a lattice shape or holes placed automatically or manually by design software on first and second shells 111a, 111b, 211a, 211b, 311a, 311b to provide air circulation, to the patient's limb. The first shell 111a, 211a, 311a is configured for mounting to the patient's limb at a first side of the articulating joint and the second shell 111b, 211b, 311b is configured for mounting to the patient's limb at a second side of the articulating joint. The latching mechanism 320a, 320b provides stability to the splint 110 relative to the joint and facilitates movement of the joint during a healing process.

In a first preferred embodiment (FIGS. 1A-1D), the splint 110 is designed as a finger orthosis with the first and second shells 111a, 11b and the pivoting joint 118 connecting the first and second shells 111a, 111b. The first shell 111a includes a first distal end 122 and a first joint end 124, wherein the first distal end 122 is spaced from the patient's joint and the first joint end 124 is positioned proximate the joint in a mounted configuration. The first shell 111a is constructed of a polymeric material and is configured to conform to the patient's limb proximate the first bone. The first shell 111a is not limited to being constructed of a polymeric material but is preferably constructed of the polymeric material and formed based on a 3D model developed based on a 3D scan of the patient's limb, as is described in further detail herein. The first shell 111a may alternatively be constructed of a metal, wooden or other relatively strong and stiff material that may be formed and configured into the preferred size and shape of the first shell 111a, withstand the normal operating conditions of the first shell 111a and perform the preferred functions of the first shell 111a, as is described herein. In the first preferred embodiment, the first shell 111a is designed and configured to have a nearly tubular U-shape that is configured to mount on one of the patient's distal and/or middle phalanges but is not so limited and may be otherwise designed and configured. The first distal end 122 is substantially closed and the first joint end 124 is substantially open and accepts insertion of the patient's phalanges therein. The first bone of the first preferred embodiment of the splint 110 is, accordingly, one or more of the patient's phalanges and the first shell 111a may be positioned on the distal and/or middle phalanges in the mounted configuration. The first shell 111 a is formed based on the 3D model of the patient's limb developed based on a 3D scan of the patient's limb.

The first shell 111a of the first preferred embodiment may have a lattice design that is configured to provide air circulation to the patient's skin in the mounted configuration. The lattice design with the integrates holes and the side openings or slots permits airflow to the patient's skin to improve comfort and potential relief to skin irritation or trauma, such as burns, abrasions or other damage to the patient's skin on the limb.

The second shell 111b has a second distal end 126 and a second joint end 128, wherein the second joint end 128 is positioned proximate the joint and the second distal end 126 is positioned opposite the second joint end 128 distal from the joint in the working configuration. The second shell 111b is configured to mount to the patient's limb proximate the second bone and proximate the joint. The first and second shells 111a, 111b are mounted to the patient's limb such that the joint is positioned proximate and between the first and second joint ends 124, 128 in the working configuration. In the first preferred embodiment, the second shell 111b is removably mountable to the proximal phalanges but is not so limited and may be otherwise designed and configured to be secured and/or mounted to another portion of the patient's body and relative to a different joint. The first preferred splint 110 may also be designed and configured for mounting to the patient's thumb with the first shell 111a mounted to the distal phalanx and the second shell 111b mounted to the proximal phalanx in the mounted configuration.

The second shell 111b of the first preferred embodiment may be constructed of a polymeric material and is configured to conform to the patient's limb proximate the second bone, which is the middle and/or proximal phalanges. The second shell 111b may be formed based on the 3D model developed based on the 3D scan of the patient's limb, as is described herein. The first preferred second shell 111b is comprised of a first shell part 130 and a second shell part 132 that are removably mountable to each other and may be pivotable and/or movable relative to each other when assembled to provide movement constraint and to provide resistance or limitations to movement of the patient's limb and joint in the working configuration.

The first preferred splint 110 also includes a pivoting joint 118 connected to the first and second shells 111a, 111b between the first and second joint ends 124, 128. The pivoting joint 118 is configured to permit controlled articulation of the joint and pivoting of the first shell 111a relative to the second shell 111b in the mounted configuration. The pivoting joint 118 preferably permits the first shell 111a to pivot relative to the second shell 111b about a pivot axis 118a to facilitate articulation of the joint during recovery, rehabilitation or any situation where articulation of the joint is desirable for patient recovery. The pivoting joint 118 may limit a range of motion of the first shell 111a relative to the second shell 111b and, therefore, the range of motion of the joint to protect the joint or otherwise facilitate healing and rehabilitation of the joint during recovery. As a non-limiting example, the pivoting joint 118 may limit pivoting of the first shell 111a relative to the second shell 111b about the pivot axis 118a to approximately ten degrees (10°) and/or may provide resistance to pivoting, but allow pivoting, around the pivot axis 118a if a resistance force is overcome by the patient. The pivoting joint 118 may also generally limit movement at the joint to pivoting to generally limit off-axis movement of the limb at the joint to prevent dislocation or damage of the joint during healing.

The first preferred system of the splint 110 also includes a central processor or artificial intelligence processing unit 116 in communication with a sensor 134 that is mounted to the splint 110, preferably to the first or second shell 111a, 111b or the pivoting joint 118. The sensor 134 collects data related to movement of the joint and transmits the data to the central processor 116. The sensor 134 is not limited to collecting data regarding movement of the joint and may collect data related to other features of the splint 110, the joint or the patient's limb, as is described in further detail herein. The sensor 134 may, for example, be comprised of a gyroscope, a goniometer, an accelerometer, a pressure sensor, a displacement sensor, a piezo sensor, a location sensor, an ultrasonic sensor, a contact sensor, a proximity sensor, a vibration sensor, an infrared sensor, a motion sensor, a displacement sensor, a microwave sensor, a velocity sensor, a wearable force sensor or another sensor that is able to sense motion or other parameters related to the splint 110, the limb, the patient's condition and/or the joint. In addition, the sensor 134 may be comprised of a plurality of sensors 134 mounted at various locations on or around the splint 110 to collect data and transmit the date to the central processor 116 for analysis and potentially to transmit instructions to the splint 110 to modify the function or operation of the splint 110 based on analysis of the collected data.

The splint 110 may include a pressure sensor 134 mounted on the first shell 111a, preferably on an inside surface of the first shell 111a proximate the patient's skin. The pressure sensor 134 collects pressure data between the first shell 111a and the patient's skin and transmits the collected pressure data to the central processor 116. The central processor is configured to monitor fit of the first shell 111a relative to the limb based on the pressure data by comparison to the collected data to a predetermined pressure range and to monitor changes in pressure. Changes in pressure, either up or down may indicate to a care provider that the limb is experiencing increased or decreased swelling based on the collected pressure data. The first shell 111a is not limited to inclusion of the pressure sensor 134 or to the specified use of the pressure sensor data and may be designed and configured without the pressure sensor 134. In addition, the pressure sensor 134 is not limited to being included only on the first shell 111a and may be mounted to the second shell 111b or both the first and second shells 111a, 111b may include pressure sensors 134 to sense pressure, collect pressure data and transmit the collected pressure data to the central processor 116.

The pivoting joint 118 may be in communication with the central processor 116 such that the central processor 116 may modify the range of motion of the pivoting joint 118. The range of motion of the pivoting joint 118 is not limited to being controllable by the central processor 116 and may be manually modifiable to change the range of motion or may be configured such that the range of motion of the pivoting of the first shell 111a relative to the second shell 111b about the pivot axis 118a is predetermined and may not be modified. The range of motion may be modified by a medical professional to facilitate physical therapy for the limb or the prevent unwanted movement of the limb.

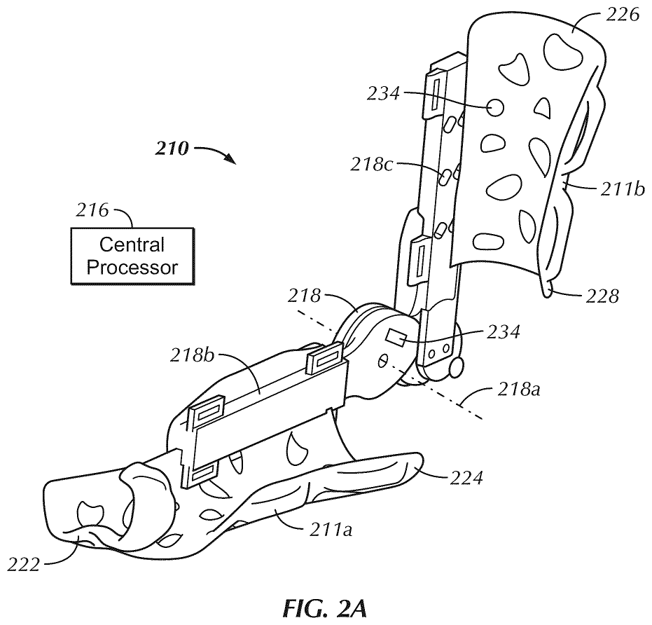

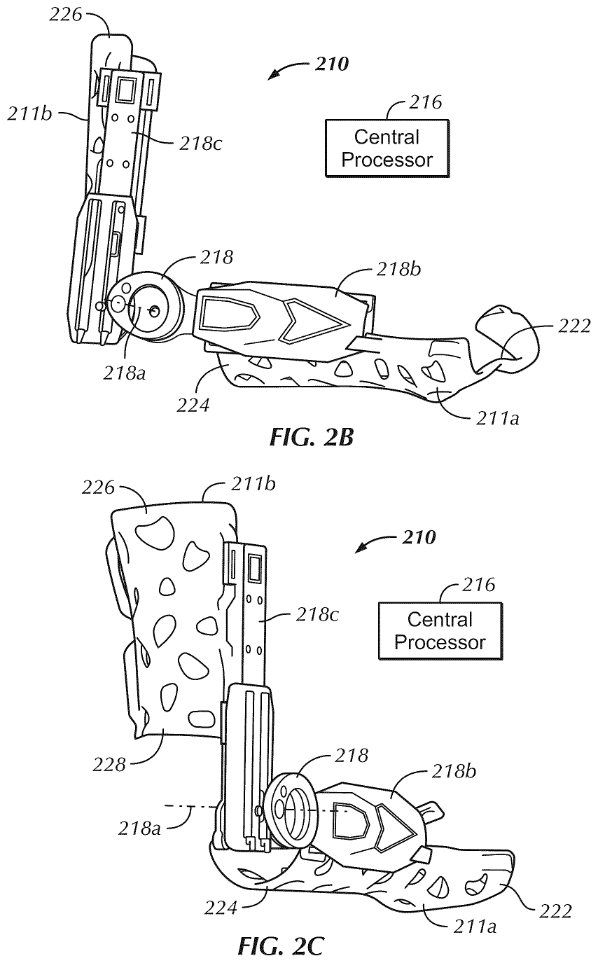

Referring to FIGS. 2A-2C, a second preferred embodiment of the splint 210 includes similar features and construction when compared to the first preferred embodiment of the splint 110. The same reference numerals are utilized to identify similar features of the second preferred embodiment when compared to the first preferred embodiment with a “2” prefix to distinguish the second preferred embodiment of the splint 210 from the first preferred embodiment of the splint 110.

The second preferred splint 210 includes the first and second shells 211a, 211b constructed of a polymeric material and having the latticed design configured to provide air circulation, reduced weight and water resistance for the patient's convenience, comfort and healing. The first and second shells 211a, 211b are configured to conform to the patient's limb proximately the first and second bones and the articulating joint, respectively. The first and second shells 211a, 211b of the second preferred embodiment are preferably formed based on the 3D model developed based on the 3D scan of the patient's limb. The first and second shells 211a, 211b are mounted to the patient's limb such that the joint is positioned proximate and between the first joint end 224 and the second joint end 228 in the working configuration.

The pivoting joint 218 of the second preferred embodiment is connected to the first and second shells 211a, 211b between the first and second joint ends 224, 228 and the pivoting joint 218 is configured to permit controlled articulation of the joint and the pivoting of the first shell 211a relative to the second shell 211b in the mounted configuration. The pivoting joint 218 of the second preferred embodiment includes a first arm 218b fixed to the first shell 211a and a second arm 218c fixed to the second shell 211b to securely attach the pivoting joint 218 to the first and second shells 211a, 211b. The first and second arms 218b, 218c are preferably constructed of a strong, stiff material to increase stability of the second preferred splint 210 and to limit movement and pivoting of the limb and articulating joint in any plane or direction, except for pivoting relative to the pivot axis 218a.

The second preferred system of the splint 210 includes the central processor 216 that is in communication with the sensor 234 to monitor movement or other parameters of the splint 210 and the limb. The sensor 234 is preferably mounted to the first or second shell 211a, 211b or the pivoting joint 218. The splint 210 of the second preferred embodiment may include a plurality of sensors 234 that are designed and configured to collect and transmit data to the central processor 216. The second preferred first and second shells 211a, 211b have generally open shapes with the latticed configuration and having a generally half cylinder configuration such that the first and second shells 211a, 211b may be quickly positioned against the patient's limb for securing to the limb, such as by taping, clamping, strapping or otherwise securing to the patient's limb. The sensors 234 mounted to the second preferred splint 210 preferably collect data related to movement of the joint but are not so limited and may not collect movement data and may collect other variables or features of the splint 210 or may not collect data without significantly impacting the operation of the splint 210. The sensors 234 may be comprised of an accelerometer, a velocity sensor, a motion sensor and/or a displacement sensor. The central processor 216 may be configured to receive data from the sensor 234, such as acceleration, elevation, velocity, force, orientation, pressure and displacement. The central processor 216 is preferably configured to utilize the data to adjust the pivoting joint 218 based on preferences of the medical provider to facilitate healing of the articulating joint and the limb.

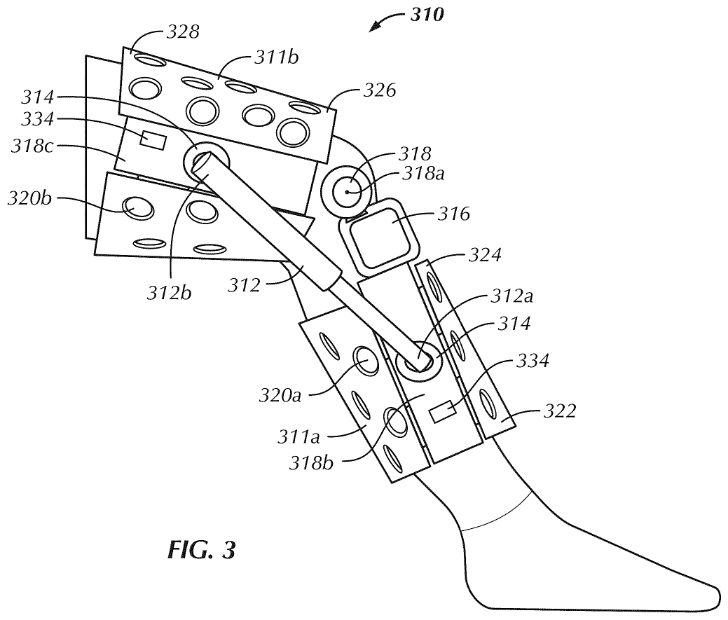

Referring to FIG. 3, a third preferred embodiment of the splint 310 includes similar features and construction when compared to the first and second preferred embodiment of the splint 110, 210. The same reference numerals are utilized to identify similar features of the third preferred embodiment when compared to the first and second preferred embodiments with a “3” prefix to distinguish the third preferred embodiment of the splint 310 from the first and second preferred embodiments of the splint 110, 210.

In a non-limiting third preferred embodiment the splint 310 is comprised of a knee splint 310. The splint 310 may be designed and configured with a hydraulic assist 312, sensors 334, such as a gyroscope 314, a goniometer, an accelerometer, a pressure sensor, a displacement sensor, a piezo sensor and/or a wearable force sensor, and the central processing unit or an artificial intelligence (“AI”) processing unit 316 for stabilization or therapy for a patient. The central processor 316 may also be designed and configured to communicate with a patient's personal fitness tracking device that may include one or more of the sensors 334 that may collect data related to how often the patient stands, how much the patient moves, how far the patient walks or runs, the number minutes the patient exercises, the amount of time the patient sleeps, the amount of energy the patient expends and related activities and data. The central processor 316 may transmit messages to the patient's personal fitness tracking device regarding warnings, alerts, suggestions or other messages related to whether the patient is following the predetermined rehabilitation program, suggestions to avoid particular movements, suggestions to schedule an appointment with their physical therapist or healthcare provider, upcoming rehabilitation schedules or programs and related messages directing the patient through their recovery or therapy. The third preferred knee splint 310 may be configured to allow for knee flection but only to ninety degrees (90°) or the limit the range of motion of the pivoting joint 318 about the pivot axis 318a. If the patient is walking and the sensors 334, 314 detect that the knee is buckling and patient is about to fall, the hydraulic assist 312 may be actuated by the processing unit 316 to rebalance the limb, so the patient doesn't fall.

The third preferred splint 10 includes the second 3D printed shell or portion 311b and the second 3D printed shell or portion 311b with first and second inflatable bladders 320a, 320b that are designed and configured to provide optimum fit to the patient's limb, such as the thigh and shin of the leg, respectively. The first bladder 320a is preferably positioned inside the first shell 311a and the second bladder 320b is preferably positioned inside the second shell 311b. The bladders 320a, 320b are preferably, selectively inflatable and configured to conform to the limb for a snug fit for the patient. The first and second bladders 320a, 320b may be inflated and deflated based on signals from the central processor 316 that may be modified based on data collected by the sensors 334. The second or upper and first or lower shells 311a, 311b are preferably connected to the pivoting joint 318 with strong and stiff support arms 318b, 318c extending from the pivoting joint 318 to attach to the first and second shells 311a, 311b and for mounting to ends of the hydraulic assist 312. The strong and stiff supports or arms 318b, 318c also preferably support the AI processing unit or central processor 316 for control of the preferred splint 310. The sizing, shape and configuration of the splint 310 is preferably optimized by the method for manufacturing the splint 310 described herein, such as sizing and shape of the first and second shells 311a, 311b, the positioning of the pivoting joint 318, the selection and control of the hydraulic assist 312 and the sizing and positioning of the supports or first and second arms 318b, 318c extending from the pivoting joint 318 to connect to the first and second shells 311a, 311b and the hydraulic assist 312. The hydraulic assist 312 may be configured to limit pivoting of the first shell 311a relative to the second shell 311b in a pre-set arc of motion. The splint 310 is not limited to including the inflatable bladders 320a, 320b and may be designed and configured without the bladders 320a, 320b without significantly impacting the configuration and function of the splint 310.

For patients who have weak limbs, the 3D printed orthosis or splints 110, 210, 310 can add “muscle strength” to help motion and/or to stabilize the knee or any other joint during use.

The preferred splint 310 of the third preferred embodiment may include an electric motor (not shown) that is controlled by the central processor 316 that controls orientation and speed control. The electric motor may include a manual movement servo and one that is programmed with commands such as acceleration, breaking, speed and like features that may be manually controlled or automatically controlled by the central processor 316. The hydraulic assist 314 preferably operates and functions with various parameters and may be adjusted and controlled in-person or by remote commands based on continued improvement in the patient's recovery like increasing/decreasing range of motion, increasing/decreasing strength assist in the hydraulics and related parameters.

The processing unit or central processor 316 on the preferred splint 310 may be in communication with a remote processing or control unit 316 that transmits parameters and control limitations to the processing unit 316 on the splint 310 to modify a patient's treatment, such as range of motion, physical therapy parameters and related limitations and parameters. The AI processing unit or central processor 316 may be configured to interface with a physical therapy telemedicine platform, such as MDLive or other healthcare applications or systems. The central processor 316 may be mounted to the pivoting joint 318, may be mounted to the first or second shells 311a, 311b or may be located remote from the splint 310. The preferred systems may, accordingly, incorporate telemedicine functionality and remote controlling for therapy of a joint, such as a leg, hip, ankle, elbow, wrist, shoulder, spine or other joint. The system may also incorporate fall detection, utilizing accelerometers and other sensors, such that the AI processing unit or central processor 316 may adjust the splint or brace 310 to provide added stability to the patient's joint.

The preferred system may be designed and configured such that the central processor 116, 216, 316 analyzes the data collected from the sensors 134, 234, 334, 314, such as the patient's motion data and additional data, and analyzes the collected data using AI tools. The central processor 116, 216, 316 may send messages to the splint 110, 210, 310 to limit or change a range of motion of the splint 110, 210, 310 based on the analysis of the data. For example, the central processor 316 may analyze data from the sensors 334, 314 and send instructions to the hydraulic assist 312 to limit the patient's range of motion, expand the patient's range of motion, increase a resistance on the joint within the patient's range of motion, decrease a resistance on the joint within the patient's range of motion or otherwise direct the splint 110, 210, 310 based on AI analysis of the data and learning based on the data collected from the patient and/or related patients who are similar to the current patient and have successfully rehabilitated their joint with a similar splint.

The central processor 316 may be remote from the patient or may be mounted to the first or second shells 311a, 311b or the pivoting joint 318 for communicating with the sensors 314, 334 and/or controlling the motor.

The hydraulic assist 312 preferably includes a first end 312a connected or mounted to the first shell 311a and a second end 312b connected or mounted to the second shell 311b. The first end 312a may also be pivotably mounted to the first arm 318b and the second end 312b may be pivotably mounted to the second arm 318c to transfer forces directly into the relatively strong and stiff first and second arms 318b, 318c and the pivoting joint 318.

The central processor 316 of the third preferred embodiment preferably received data collected from the sensors 314, 334 and is configured to actuate the hydraulic assist 312 to rebalance the limb to limit patient instability. The central processor 316 is not limited to being configured to enhance patient stability but may be so configured or may be otherwise configured to facilitate healing of the articulating joint, to enhance therapy of the limb or to otherwise improve healing or comfort of the patient.

The hydraulic assist 312 in combination with the pivoting joint 318 of the third preferred embodiment may have a pre-set arc of motion or range of motion that can be adjusted manually or automatically with the central processor 316.

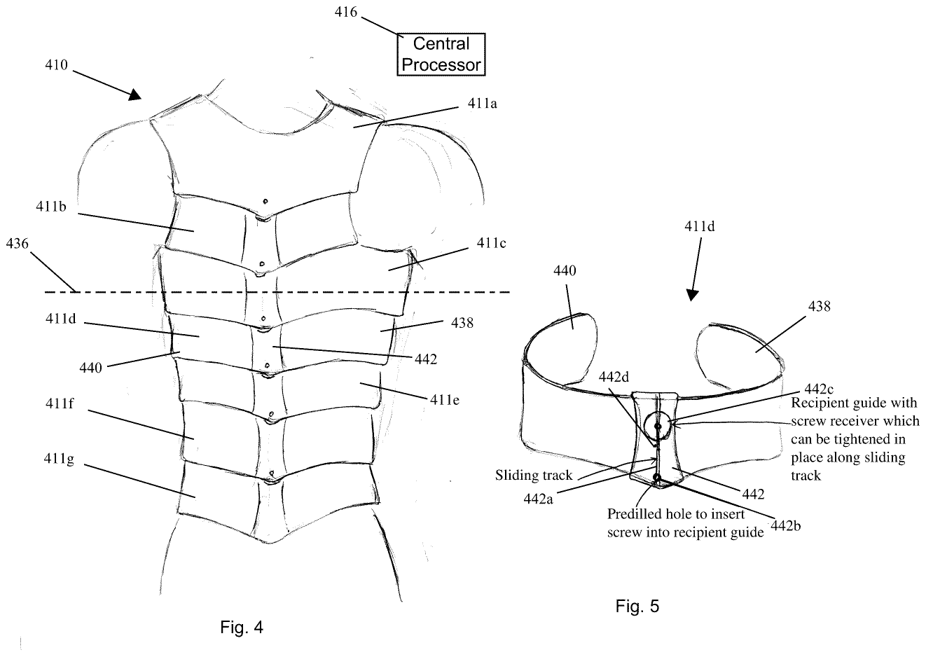

Referring to FIGS. 4 and 5, a fourt preferred embodiment of the splint or brace 410 includes similar features and construction when compared to the first, second and third preferred embodiments of the splint 110, 210, 310. The same reference numerals are utilized to identify similar features of the fourth preferred embodiment when compared to the first, second and third preferred embodiments with a “4” prefix to distinguish the fourth preferred embodiment of the splint 410 from the first, second and third preferred embodiments of the splint 110, 210, 310.

The fourth preferred splint or brace 410 is preferably designed and configured to splint, brace and support a patient's spine. The brace 410 includes a plurality of shell or ring portions 411a, 411b, 411c, 411d, 411e, 411f, 411g that are preferably individually designed and configured to mount to various portions of the patient's trunk to support and allow limited articulation of the patient's spine. The first portion 411a is preferably designed to mount to and support the spine proximate the patient's shoulders, the second portion 411b is designed to mount between the patient's shoulder blades and the third, fourth, fifth, sixth and seventh portions 411c, 411d, 411e, 411f, 411g are designed to mount from below the patient's shoulder blades to a lumbar portion of the patient's spine. The first through seventh portions 411a, 411b, 411c, 411d, 411e, 411f, 411g are not limited to being designed and configured as shown to mount and support the specific portions of the patient's spine shown in FIG. 4 and may be designed and configured to support additional portions of the patient's spine, such as the neck or cervical spine or may be designed and configured to support only smaller or more limited portions of the patient's spine.

Each of the first through seventh portions 411a, 411b, 411c, 411d, 411e, 411f, 411g of the spinal support splint or brace 410 is designed to articulate relative to adjacent portions 411a, 411b, 411c, 411d, 411e, 411f, 411g, creating a harmonious and functional alignment that mirrors the natural structure of the human spine. This articulation of the first through seventh portions 411a, 411b, 411c, 411d, 411e, 411f, 411g is designed to provide not only support but also flexibility, allowing for a range of movements that are desired for the patient's daily activities. The fourth preferred spine splint's 410 configuration facilitates bending or articulation of the patient's spine about a horizontal axis, while limiting bending or articulation around sagittal and longitudinal axes, thereby stiffening and providing limited mobility of the spine to promote healing in the mounted configuration (FIG. 4).

Each of the first through seventh portions 411a, 411b, 411c, 411d, 411e, 411f, 411g have similar designs and configurations and the fourth preferred portion 411d is described herein and shown in FIG. 5 as an example of the portions 411a, 411b, 411c, 411d, 411e, 411f, 411g with the understanding that the additional portions 411a, 411b, 411c, 411e, 411f, 411g have similar designs with configurations to fit the patient's trunk at the associated location of the patient's body. The fourth preferred portion 411d includes first and second arms 438, 440 that extend outwardly from a central support 442. The central support 442 is preferably positioned proximate the patient's spine and the first and second arms 438, 440 extend from the central support 442 and wrap around a portion of the patient's trunk to engage the patient's trunk and secure the fourth preferred splint 410 to the trunk. The first and second arms 438, 440 are not limited to being designed and configured as shown for the fourth ring portion and may be otherwise designed and configured to secure the splint 410 to the patient's trunk to facilitate the operation of the splint 410, as described herein, and immobilize and allow limited articulation and bending of the spine, as desired by the healthcare professional. The first and second arms 438, 440 are preferably individually designed for mounting to the specific portions of the patient's trunk, such as the arms of the first portion 411a extending upwardly from the central support and over the patient's trapezius, the arms of the second portion 411b being relatively short to provide relief for movement of the patient's arms and the additional first and second arms of the third, fourth, fifth, sixth and seventh portions 411c, 411d, 411e, 411f, 411g having generally U-shaped that wrap around the patient's sides with different lengths and curvature to conform generally to the patient's anatomy.

The central support 442 preferably includes a longitudinal sliding track 442a, an engagement hole 442b extending through a portion of the sliding track 442a, a pin with a head 442d on an inside portion of the central support 442 and a recipient guide 442c. The sliding track 422a is comprised of an undercut groove on a rear side of the central support 442 that is designed and configured to dictate degrees of flexion and extension of the patient's spine in the mounted configuration. The sliding track 442a with the pin with the head 442d from an adjacent ring portion 411a, 411b, 411c, 411d, 411e, 411f engaged therein is designed and configured to guide and limit the spine's movement while simultaneously offering support and stability. In the mounted configuration, the pin with the head 442d is slidably positioned in the sliding track 442a of the adjacent inner ring portion 411b, 411c, 411d, 411e, 411f, 411g to guide and limit movement of the ring portions 411a, 411b, 411c, 411d, 411e, 411f, 411g relative to each other. The recipient guide 442c provides a stop for the pin with the head 442d and spacing between adjacent ring portions 411a, 411b, 411c, 411d, 411e, 411f, 411g in the mounted configuration. The recipient guide 442c may be fastener mounted to the adjacent central support 442 by a screw or bolt that engaged the sliding track 442a or other portion of the central support 442 to fix the recipient guide 442c to the central support 442. The recipient guide 442c may be engaged nearly anywhere along the sliding track 442a to modify the operation and configuration of the joint between the adjacent ring portions 411a, 411b, 411c, 411d, 411e, 411f, 411g and limit motion of the patient's spine. The recipient guide 442c may also be designed and configured to act as a spring and damper to facilitate movement of the patient's spine that is resisted and controlled by the spine splint 410 to promote patient therapy and healing. By controlling the extent of forward and backward bending, the spine splint 410 may be configured such that the spine remains within safe and therapeutic limits, thereby aiding in recovery or managing chronic conditions using a controller knob 403. The engagement hole 442b is preferably designed to facilitate assembly of adjacent ring portions 411a, 411b, 411c, 411d, 411e, 411f, 411g by accepting the head of the pin with the head 422d from the adjacent ring portion 411b, 411c, 411d, 411e, 411f, 411g into the sliding track 442a.

The size, shape and configuration of the sliding track 442a and the pin with the head 422d is preferably designed and configured to control or limit the lateral bending and rotation of the spine. This feature is preferred to allow for a controlled range of motion in multiple planes, which facilitates healing for the complex dynamics of spinal movement. The design and configuration of the spine splint 410 may be further enhanced by the incorporation of adjustable stops (not shown) within the grooves. These stops are strategically placed to limit the excursion of the grooves, thereby preventing excessive movement that could potentially be harmful. This aspect of design not only adds a layer of safety but also allows for customization according to individual patient needs. Whether restricting certain movements post-surgery or providing specific support for degenerative spinal conditions, these adjustable stops tailor the spine splint 410 to provide optimal support and protection, making it a versatile tool in spinal care and rehabilitation.

Additionally, the spine splint 410 preferably integrates an advanced motion control mechanism, programmable and automated based on the patient's initial imaging data and morphological analysis that is controlled and tracked by the central processor 416. This sophisticated feature allows for the customization of the splint's 410 movement patterns and alignment, tailored specifically to each patient's unique spinal structure and condition, thereby enhancing the efficacy and personalization of spinal splint 410. The central processor 416 also preferably collects data regarding the movement and function of the spine splint 410 during use to track the patient's healing and rehabilitation. The central processor 416 is preferably in communication with sensors (not shown) on the spine splint 410 to collect data regarding the operation of the spline splint during use, similar to the data collection and control features of the above-described first, second and third splints 110, 210, 310. The fourth preferred splint 410 may include the hydraulic assist, gyroscope, latching mechanism including the inflatable bladders, sensors and other components to assist with collecting data and controlling the spine splint 410.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1-36. (canceled)

37. A spine splint for supporting a patient's spine and facilitating limited movement of the patient's spine, the spine splint comprising:
a series of interconnected ring portions including a first ring portion and a second ring portion, the first ring portion configured to articulate relative to the second ring portion, the first ring portion movably engaged to the second ring portion by a pin slidably positioned within a sliding track thereby mimicking a natural articulation of the patient's spine, the pin positioned on an inside portion of the first ring portion and the sliding track positioned on an outside portion of the second ring portion on a second central support, the second central support positioned between first and second arms of the second ring portion, the first and second arms configured to wrap around a portion of a trunk of the patient to engage the patient's trunk and secure the splint to the trunk.

38. The spine splint of claim 37, wherein the first ring portion includes a first central support, the pin comprised of a first pin extending from an inner portion of the first central support, the sliding track comprised of a second sliding track defined in the second central support of the second ring portion, the engagement of the first pin in the second sliding track and the configuration of the first and second ring portions governing flexion and extension of the first ring portion relative to the second ring portion in a mounted configuration, thereby at least partially controlling forward and backward bending movements of the patient's spine.

39. The spine splint of claim 38, wherein a shape of the second sliding track and the first pin are configured to determine a lateral bending and rotational movement of the first ring portion relative to the second ring portion, thereby allowing for controlled multi-planar spinal motion.

40. The spine splint of claim 39, wherein the second sliding track includes adjustable stops, the stops configured to limit a range of motion of the first ring portion relative to the second ring portion to limit movement and enhance spinal stability in the mounted configuration.

41. The spine splint of claim 40, wherein the adjustable stops are customizable and configured to tailor the spine splint to individual patient needs and anatomy, the adjustable stops configured to limit movement to enhance post-surgical rehabilitation and support for degenerative spinal conditions of the spine.

42. The spine splint of claim 37, wherein the series of interconnected ring portions are constructed from a material that provides both flexibility and support, the series of interconnected ring portions configured for comfort and structural integrity.

43. The spine splint of claim 42, wherein the material is selected from a group consisting of medical-grade plastics, metals, and composite materials.

44. The spine splint of claim 37, further comprising:
an automated motion adjustment system, wherein a motion of each of the interconnected ring portions is automatically adjusted based on initial imaging data and morphological analysis of the patient's spine, the automated motion adjustment system configured to provide a customized fit and movement pattern specific to the patient's anatomical and physiological requirements.

45. The spine splint of claim 44, wherein the automated motion adjustment system utilizes advanced algorithms to interpret spinal imaging data to determine an optimal range of motion and alignment for each of the interconnected ring portions relative to each other, thereby ensuring conformity to a patient's spinal curvature and condition.

46. The spine splint of claim 45, wherein the spinal imaging data is collected from magnetic resonance imaging (“MRI”) scanning or computerized tomography (“CT”) scanning,

47. The spine splint of claim 45, wherein the automated motion adjustment system is configured to dynamically adjust movements of the series of interconnected ring portions at predetermined intervals to adapt to changes in a patient's condition or as part of a prescribed rehabilitation plan.

48. The spine splint of claim 47, wherein adjustments made by the automated motion adjustment system include changes to a position of adjustable stops within the sliding track, thereby modifying an extent of flexion, extension, lateral bending, and rotation permitted by the spine splint.

49. The spine splint of claim 48, further comprising:
a user interface configured to allow a healthcare provider to input a rehabilitation plan or adjustments based on ongoing patient assessments, thereby enabling the spine splint to adapt to evolving therapeutic needs.

50. The spine splint of claim 49, wherein the user interface is integrated with a healthcare management system, allowing for seamless updates and monitoring of the patient's progress and facilitating adjustments to the spine splint based on real-time feedback and data collection from sensors communicating with a central processor.

51. The spine splint of claim 50, wherein the automated motion adjustment system and user interface are wirelessly connected to external devices, the central processor configured to remotely monitor and adjust the spine splint based on data collected from the external devices.

52. A method of supporting a spine of a patient using the spine splint of claim 40, the method comprising:
fitting the spine splint, including the series of interconnected ring portions to the spine; and
adjusting the adjustable stops in the second sliding track to limit the range of motion.
